(19) United States
(12) Patent Application Publication
(10) Pub. No.: US 2016/0361326 A1
(43) Pub. Date: Dec. 15, 2016

(54) METHOD OF TREATING HYPERGLYCEMIA AND DIABETES AND OF INCREASING ADSORPTION AND EFFICACY OF VITAMIN D

(71) Applicant: Sushil Kumar JAIN, Shreveport, LA (US)

(72) Inventor: Sushil Kumar JAIN, Shreveport, LA (US)

(73) Assignee: Board of Supervisors of Louisiana State University and Agricultural and Mechanical College, Baton Rouge, LA (US)

(21) Appl. No.: 15/178,751

(22) Filed: Jun. 10, 2016

Related U.S. Application Data
(60) Provisional application No. 62/174,806, filed on Jun. 12, 2015.

(51) Int. Cl. A61K 31/593; A61K 31/195; A61K 45/06; A61K 31/198

(52) U.S. Cl. CPC A61K 31/593; A61K 31/198; A61K 31/195; A61K 45/06

(57) ABSTRACT

A pharmaceutical for and a method of treating one of vitamin D deficiency, diabetes and pre-diabetes in an animal comprising the steps A method of treating one of vitamin D deficiency, diabetes and pre-diabetes in an animal comprising the steps of administering a first therapeutic agent and a second therapeutic agent to the animal; providing the first therapeutic agent is one of vitamin D, a pharmaceutically acceptable salt, solvate, clathrate, stereoisomer, enantiomer or prodrug thereof, and a pharmaceutically acceptable derivative thereof; and providing the second therapeutic agent is one of L-cysteine, glutathione; glycine, glutamate, and glutathione, or a pharmaceutically acceptable salt, solvate, clathrate, stereoisomer, enantiomer or prodrug thereof, pharmaceutically acceptable derivative thereof, or some combination of thereof.

### CROSS REFERENCE TO RELATED APPLICATIONS/PRIORITY

The present invention claims priority to U.S. Provisional Patent Application No. 62/174,806 filed Jun. 12, 2015, which is incorporated by reference into the present disclosure as if fully restated herein. To the extent that there is any conflict between the incorporated material and the present disclosure, the present disclosure will control

### FIELD

The present invention relates generally to methods and products for increasing adsorption and efficacy of vitamin D and to treatments of diabetes and vitamin D deficiency.

### BACKGROUND

Vitamin D deficiency has become a world-wide epidemic, particularly affecting African Americans. African Americans have the highest rate of microvascular complications associated with diabetes and the highest incidence of vitamin D deficiency. Vitamin D deficiency has been implicated in the excessive rate of complications associated with diabetes in African Americans. Blood levels of Vitamin D binding protein and glutathione are lower in African Americans compared with those in Caucasians. However, vitamin D supplementation alone is only marginally effective in increasing vitamin D levels in the blood. Currently, for individuals with vitamin D deficiency, high dosages of vitamin D supplementation are needed to raise vitamin D concentration in blood to the required level, but this comes at a cost. First, in addition to raising vitamin D concentration, high dosages of vitamin D supplementation do not appear to convey the desired health outcome of addressing problems associated with vitamin D deficiency. Additionally, high dosages of vitamin D have negative and sometimes harmful side effects, such as hypercalcemia and other complications. There is no product in the art that is known to increase the efficacy of vitamin D and help increase the blood levels of vitamin D at low or moderate dosages of vitamin D supplementation.

### SUMMARY

Wherefore, it is an object of the present invention to overcome the above mentioned shortcomings and drawbacks associated with the prior art.

The inventors have observed a link between vitamin D and vitamin D binding protein (VDBP) status and better health. Vitamin D binding protein is a transporter of vitamin D, protects against vitamin D deficiency, and is a novel regulator of vitamin D action and metabolism. Both humans with a genetic mutation for vitamin D binding protein and vitamin D binding protein-knockdown mice demonstrate low plasma levels of vitamin D. Genetic variations in vitamin D binding protein influence vitamin D blood levels in response to vitamin D supplementation. Vitamin D binding protein is produced by monocytes and by the liver. Vitamin D binding protein is a 52-59 kDa monomeric glycoprotein with a short half-life, 2.5 to 3 days compared with the 1-2 months for vitamin D. Other factors such as age, sex, skin pigmentation, season, geographic latitude, food and supplemental sources of vitamin D, and adiposity also influence the vitamin D status in the human body. It has been thought that dark pigmentation of the skin absorbs much of the UV B energy before it has an opportunity to stimulate production of vitamin D, to which is attributed the higher rates of vitamin D deficiency observed in African Americans. Vitamin D deficiency has been associated with an increased risk of atherosclerosis, carotid artery thickness, and cardio vascular disease. This has led to the practice of widespread vitamin D supplementation. However, alone, supraphysiological doses of vitamin D alone are required to reach desired concentrations of circulating vitamin D. However, such high doses of vitamin D can cause undesirable elevated blood levels of calcium, which leads to vascular and tissue calcification, with subsequent damage to the heart, blood vessels, and kidneys. An object of the present invention is do disclose a therapy to upregulate vitamin D binding protein with lower doses of vitamin D supplementation to achieve a desired circulating vitamin D level.

As disclosed herein, the inventors showed that among other things, lower glutathione levels impact vitamin D binding protein and vitamin D deficiency in African Americans type 2 diabetic patients.

In further experimentation, the inventors further showed that L-cysteine supplementation upregulated glutathione (GSH) status in an FL83B hepatocyte cell culture model and in vivo using Zucker diabetic fatty (ZDF) rats. The L-cysteine supplementation upregulated both protein and mRNA expression of vitamin D binding protein and vitamin D receptor (VDR) and glutathione status in hepatocytes exposed to high glucose, and that glutathione deficiency, induced by glutamate cysteine ligase knockdown, resulted in the downregulation of glutathione, vitamin D binding protein, and vitamin D receptor and an increase in oxidative stress levels in hepatocytes. In vivo, L-cysteine supplementation increased glutathione and protein and mRNA expression of vitamin D binding protein and vitamin D 25-hydroxylase (CYP2R1) in the liver, and simultaneously resulted in elevated blood levels of L-cysteine and glutathione, as well as increases in vitamin D binding protein and 25(OH) vitamin D levels, and decreased inflammatory biomarkers in ZDF rats compared with those in placebo-supplemented ZDF rats consuming a similar diet.

Another object of the present invention are treatments of vitamin D deficiency with dual L-cysteine and vitamin D supplementation or with supplementation of a bound complex of vitamin D and L-cysteine. Such treatments will increase vitamin D binding protein, which is a transporter of vitamin D, and will upregulate vitamin D receptor, which is needed for metabolic actions of vitamin D. Thus, supplementation with L-cysteine and vitamin D together will increase the blood levels of vitamin D as well actions and/or efficacy of vitamin D, thereby reducing the epidemic of vitamin D deficiency and associated poor health in the population. The combined or complex L-cysteine/vitamin D supplementation is effective at lower dosages of vitamin D supplementation, and therefore does not bring the negative side effects associated with high-dose vitamin D.

Yet another object of the present invention is to provide as part of the combined or complex L-cysteine/vitamin D supplementation, in addition to L-cysteine or as an alternative to L-cysteine, chemicals metabolically related to

L-cysteine. These chemicals include glutathione; glycine, glutamate, and glutathione, or some combination of each, including pharmaceutically acceptable salts, solvates, clathrates, stereoisomers, enantiomers or prodrugs thereof, and pharmaceutically acceptable derivatives thereof.

A still further object of the present invention is to treat diabetes with the combined or complex L-cysteine/vitamin D supplementation alone or the combined or complex L-cysteine/vitamin D supplementation in combination with one or more diabetes drugs for increased treatment efficacy. These one or more diabetes drugs include one or more of sulfonylureas, such as glyburide, glimepiride, tolbutamide, tolazamide, chlorpropamide and glipizide, biguanides, such as metformin, meglitinides, such as repaglinide and nateglinide, thiazolidinediones, such as rosiglitazone and pioglitazone, DPP-4 inhibitors, such as sitagliptin, saxagliptin, linagliptin, and alogliptin, SGLT2 inhibitors, such as canagliflozin, and dapagliflozin, alpha-glucosidase inhibitors, such as acarbose and miglitol, and bile acid sequestrants, such as colesevelam, for example, and pharmaceutically acceptable salts, solvates, clathrates, stereoisomers, enantiomers or prodrugs thereof, and pharmaceutically acceptable derivatives thereof.

A further object is to treat inflammation, including inflammation in patients with diabetes.

A further object of the present invention is to treat vitamin D deficiency.

A still further object of the present invention is to treat one of vitamin D deficiency, diabetes and pre-diabetes with combined or a complex of L-cysteine/vitamin D supplementation. The L-cysteine dosage being preferably between 1 mg and 10 mg per kg body weight per day, more preferably between 2.5 mg and 7.5 mg per kg body weight per day and most preferably substantially 5 mg per kg body weight per day. The vitamin D dosage being preferably between 50 and 1500 IU per day, more preferably between 100 and 1000 IU per day, and most preferably between 600 and 750 IU per day.

The present invention is related to substances and methods of treating one of vitamin D deficiency, diabetes and pre-diabetes in an animal comprising the steps of administering a first therapeutic agent and a second therapeutic agent to the animal, wherein the first therapeutic agent is one of vitamin D, a pharmaceutically acceptable salt, solvate, clathrate, stereoisomer, enantiomer or prodrug thereof, and a pharmaceutically acceptable derivative thereof; and wherein the second therapeutic agent is one of L-cysteine, glutathione; glycine, glutamate, and glutathione, or a pharmaceutically acceptable salt, solvate, clathrate, stereoisomer, enantiomer or prodrug thereof, pharmaceutically acceptable derivative thereof, or some combination of thereof. According to an additional embodiment, the first therapeutic agent is administered in a dosage of between 100 IU and 1500 IU. According to an additional embodiment, the second therapeutic agent is administered in a dosage of between 1 mg and 10 mg per kg of a body weight of the animal. According to an additional embodiment, the animal is a human. An additional embodiment further comprises the step of administering the first and the second therapeutic agent in a single pill or serum. An additional embodiment further comprising the step of administering a third therapeutic agent. According to an additional embodiment, the third therapeutic agent is one or more of a sulfonylurea, a biguanide, a meglitinide, a thiazolidinedione, a DPP-4 inhibitor, an SGLT2 inhibitor, an alpha-glucosidase inhibitor, and a bile acid sequestrant, and a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a sulfonylurea or a pharmaceutically acceptable salt, solvate, clathrate, stereoisomer, enantiomer or prodrug thereof, pharmaceutically acceptable derivative thereof, or some combination of thereof. According to an additional embodiment, the sulfonylurea is one of glyburide, glimepiride, tolbutamide, tolazamide, chlorpropamide and glipizide or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a biguanide from metformin or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a meglitinide from one of repaglinide and nateglinide or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a thiazolidinedione from one of rosiglitazone and pioglitazone or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a DPP-4 inhibitor from one of sitagliptin, saxagliptin, linagliptin, and alogliptin or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a SGLT2 inhibitor from one of canagliflozin and dapagliflozin or a pharmaceutically acceptable salt, solvate, clathrate, stereoisomer, enantiomer or prodrug thereof, pharmaceutically acceptable derivative thereof, or some combination of thereof. 15. The method of claim 6 wherein the third therapeutic agent is an alpha-glucosidase inhibitor from one of acarbose and miglitol or a pharmaceutically acceptable salt, solvate, clathrate, stereoisomer, enantiomer or prodrug thereof, pharmaceutically acceptable derivative thereof, or some combination of thereof. According to an additional embodiment, the third therapeutic agent is a bile acid sequestrant colesevelam or a pharmaceutically acceptable salt, solvate, clathrate, stereoisomer, enantiomer or prodrug thereof, pharmaceutically acceptable derivative thereof, or some combination of thereof. According to an additional embodiment, the vitamin D is one or more of vitamin D₁, vitamin D₂, vitamin D₃, vitamin D₄, and vitamin D₅ According to an additional embodiment, the human is African American.

A further embodiment of the present invention relates to pharmaceuticals comprising a first and a second therapeutic agent; where the first therapeutic agent is one of vitamin D, a pharmaceutically acceptable salt, solvate, clathrate, stereoisomer, enantiomer or prodrug thereof, pharmaceutically acceptable derivative thereof, or some combination of thereof; and the second therapeutic agent is one of L- cysteine, glutathione; glycine, glutamate, and glutathione, or a pharmaceutically acceptable salt, solvate, clathrate, stereoisomer, enantiomer or prodrug thereof, pharmaceutically acceptable derivative thereof, or some combination of thereof. According to an additional embodiment, the pharmaceutical further comprises a third therapeutic agent, wherein the third therapeutic agent is one or more of a sulfonylurea, a biguanide, a meglitinide, a thiazolidinedione, a DPP-4 inhibitor, an SGLT2 inhibitor, an alpha-glucosidase inhibitor, and a bile acid sequestrant, and a pharmaceutically acceptable salt, solvate, clathrate, stereoisomer, enantiomer or prodrug thereof, pharmaceutically acceptable derivative thereof, or some combination of thereof.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1F are experimental results showing relationships between different chemicals in African-American type 2 diabetic patients;

FIGS. 2A-2I are experimental results showing the effect of L-cysteine supplementation in control and high-glucose treated THP-1 monocytes and the effect of glutathione deficiency in THP-1 monocytes treated with different concentrations of glutamate cysteine ligase catalytic unit-siRNA; and

FIGS. 3A-3E are experimental results showing the effect of L-cysteine and vitamin D supplementation in control and high-glucose treated THP-1 monocytes.

FIGS. 4A-4E are experimental results showing the effect of L-cysteine supplementation in hepatocytes treated with control and high glucose. Levels of vitamin D binding protein are shown in FIGS. 4A and 4B, vitamin D receptor are shown in FIGS. 4C and 4D and glutathione in FIG. 4E in hepatocytes cultured with high glucose. Values are mean±SE, n=4.

FIGS. 5A-5H are experimental results showing show the effect of glutathione deficiency on GCLC knockdown (GCLC-KD) in FIGS. 5A and 5B, glutathione in FIG. 5C, reactive oxygen species in FIG. 5D, vitamin D binding protein in FIGS. 5E and F, and vitamin D receptor in FIGS. 5G and H levels in FL83B hepatocytes treated with different concentrations of GCLC siRNA (Mean±SE, n=4).

FIGS. 6A-6E are experimental results showing the effect of L-cysteine supplementation on levels of vitamin D binding protein (FIG. 6E), 25(OH) vitamin D (FIG. 6D), glutathione (FIG. 6C), MCP-1 (FIG. 6B), and CRP (FIG. 6A) levels in the plasma of diabetic control (D+P, n=6) and L-cysteine-supplemented (D+LC, n=6) Zucker diabetic fatty rats. Rats were supplemented with placebo (saline) or L-cysteine (1 mg/kg BW) via daily oral gavage for 8 weeks. Values are mean±SE.

FIGS. 7A-7F are experimental results showing the effect of L-cysteine supplementation on protein and mRNA expression of vitamin D binding protein (FIGS. 7A and 7B), CYP2R1 (FIG. 7D and 7E), reactive oxygen species (FIG. 7F), and glutathione (FIG. 7C) levels in the livers of diabetic control (D+P, n=6) and L-cysteine-supplemented (D+LC, n=6) Zucker diabetic fatty rats. Blots shown here are from three rats each. Rats were supplemented with placebo or L-cysteine (1 mg/kg BW) via daily oral gavage for 8 weeks. Values are mean±SE.

FIGS. 8A-8G are experimental results showing vitamin D binding protein (FIGS. 8A and 8B), vitamin D receptor (FIGS. 8C and 8D), and CYP2R1 (FIGS. 8E and 8F) expression and reactive oxygen species (FIG. 8G) levels in the livers of Zucker diabetic fatty (n=6) and age-matched normal rats (n=6). Values are mean±SE. Details of treatment, euthanasia of rats, and blood and tissue analyses are given in the Material and methods.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example at least 1″ means 1 or more than 1. The term “at most” followed by a number is used herein to denote the end of a range ending with that number (which may be a range having 1 or 0 as its lower limit, or a range having no lower limit, depending upon the variable being defined). For example, at most 4″ means 4 or less than 4, and at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention

Vitamin D (VD) deficiency and an excessive rate of diabetes has become a world-wide epidemic, particularly affecting African Americans. The inventors observed a significant positive correlation between lower vitamin D binding protein and vitamin D, L-cysteine, and glutathione blood levels in African Americans diabetic patients. The inventors further observed that raising glutathione status via L-cysteine supplementation upregulated both vitamin D binding protein and vitamin D receptor in monocytes. Vitamin D binding protein is a key determinant of vitamin D blood levels. This novel treatment will impact the clinical practice of vitamin D supplementation and the development of new therapies based on the potential of L-cysteine coupled with lower vitamin D doses to improve the efficacy and levels of vitamin D and health outcomes in African Americans.

The results of the experimentation show plasma levels of L-cysteine, glutathione, vitamin D binding protein, and vitamin D were significantly lower in African Americans with type 2 diabetes compared with age-matched healthy African Americans (AA-N) or Caucasians with type 2 diabetes. Lower levels of L-cysteine and glutathione showed a significant positive correlation with lower vitamin D binding protein and vitamin D levels in African Americans with type 2 diabetes. An investigation of glutathione deficiency used an antisense approach depleted vitamin D binding protein/vitamin D receptor. L-cysteine supplementation caused significant upregulation of glutathione and of vitamin D binding protein/vitamin D receptor, while supplementation with vitamin D+L-cysteine caused significantly greater glutathione and vitamin D binding protein/vitamin D receptor upregulation compared with that of vitamin D alone in monocytes. The disclosed observations provide foundation for the treatment of vitamin D deficiency based on glutathione and L-cysteine status, and lead to a novel therapy and therapeutic of supplementation with L-cysteine in combination with vitamin D for effectively increasing vitamin D levels and efficacy and reducing health disparities in African Americans.

### EXAMPLE 1

Regarding the relationship of L-cysteine and glutathione status on vitamin D binding protein and vitamin D levels, especially in African Americans type 2 diabetic patients, blood levels of L-cysteine, glutathione, vitamin D binding protein, and vitamin D are significantly lower in African Americans with type 2 diabetes compared to those in age-matched African Americans healthy subjects (AA-N) or age-matched Caucasians with type 2 diabetes. These levels are shown in Table I below. Variables include body weight, glucose, vitamin D binding protein, vitamin D, and L-cysteine levels in the fasting blood of type 2 diabetic patients (T2D) and age- and race-matched healthy non-diabetic subjects. Values are mean±SE. Values marked ‘*’ versus #, between # versus ##, and * versus @ are significant (p<0.05). ND: not determined. Normals are age- and race-matched healthy non-diabetic subjects. AA: African Americans; N: healthy non-diabetic subjects; T2D: type 2 diabetic patients; Caucasians: European Americans.

TABLE 1

| | AA-T2D | AA-N | Caucasians-T2D | Caucasians-N |
|---|---|---|---|---|
| N | 51 | 32 | 27 | 20 |
| Age (years) | 47.6 ± 1.3 | 47 ± 2.1 | 50.3 ± 1.6 | 46.8 ± 3.1 |
| Body Weight (Kg) | 104.2 ± 3.7* | 77.2 ± 6.5# | 102.3 ± 8.6** | 84.1 ± 4.3## |
| Glucose (mg/dL) | 139 ± 8* | 108 ± 5# | 126 ± 8** | 87 ± 4## |
| HbA₁C (%) | 7.99 ± 0.3 | ND | 7.0 ± 0.2 | ND |
| GSH (μM) | 66.1 ± 2.9* | 80.4 ± 6.8# | 79.6 ± 6.1**,^{@} | 102 ± 5.3## |
| L-cysteine (μM) | 296 ± 14* | 363 ± 26# | 342 ± 26^{@} | 398 ± 18 |
| VDBP (μg/mL) | 63.2 ± 7.2* | 156 ± 26# | 126 ± 27^{@} | 177 ± 12 |
| Vitamin D (nM) | 14.3 ± 1.1* | 18.9 ± 2.1# | 18.7 ± 2.2^{@} | 21.8 ± 2.7 |

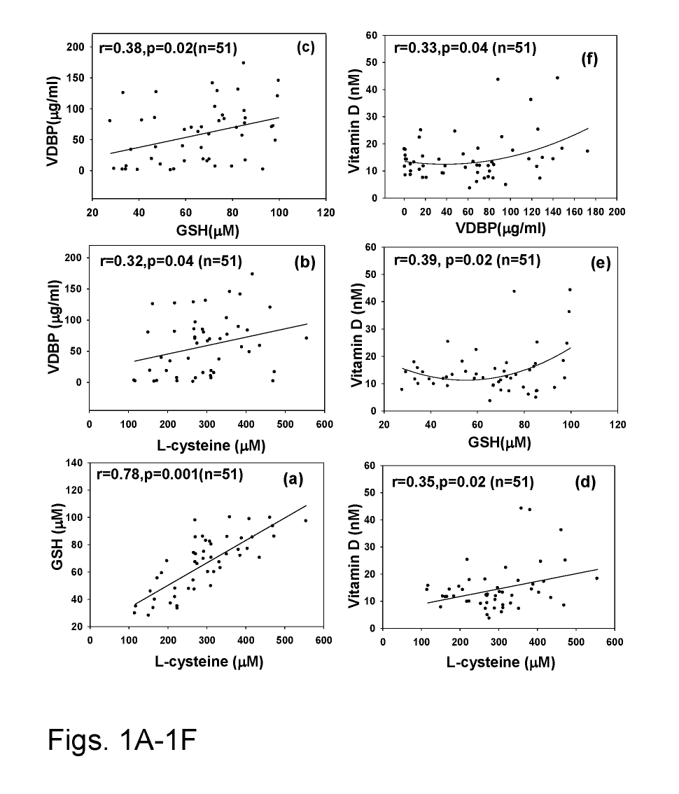

Turning now to FIG. 1, relationships between different chemicals in African-American type 2 diabetic patients are shown. Regression analyses showed a significant positive correlation between the blood levels of L-cysteine and glutathione (FIG. 1A), of L-cysteine and vitamin D binding protein (FIG. 1B), of L-cysteine and vitamin D (FIG. 1D), of glutathione and vitamin D binding protein (FIG. 1C), of glutathione and vitamin D (FIG. 1E), and of vitamin D binding protein and vitamin D (FIG. 1F) in African Americans type 2 diabetes patients. The relationship between plasma levels of L-cysteine and those of glutathione, vitamin D binding protein, and vitamin D are shown in FIGS. 1A, 1B, and 1D respectively. The relationship between plasma glutathione levels and those of vitamin D binding protein (FIG. 1C) and vitamin D (FIG. 1E) are shown in FIGS. 1C and 1E respectively. The relationship between plasma levels of vitamin D binding protein and Vitamin D is shown in FIG. 1F. Body weight was used as an additional independent variable to determine regression and p value. The data show that in type 2 diabetic patents there is a significant positive association between the blood levels of L-cysteine and those of glutathione (FIG. 1A), vitamin D binding protein (FIG. 1B) and vitamin D (FIG. 1D), between the blood levels of glutathione and vitamin D binding protein (FIG. 1C), and between the blood levels of vitamin D binding protein and vitamin D (FIG. 1F).

In analyzing the data, the first order relationships were for glutathione versus vitamin D (r=0.27, p=0.07, FIG. 1E) and vitamin D binding protein versus vitamin D (r=0.29, p=0.05, FIG. 1F). The associations were stronger when a second order relationship was calculated for glutathione versus vitamin D (r=0.39, p=0.02, FIG. 1E) and vitamin D binding protein versus vitamin D (r=0.33, p=0.04, FIG. 1F). This significant relationship between blood levels of vitamin D binding protein, vitamin D, and those of glutathione and L-cysteine provides insight and support for the disclosed new modes of treatment for vitamin D deficiency.

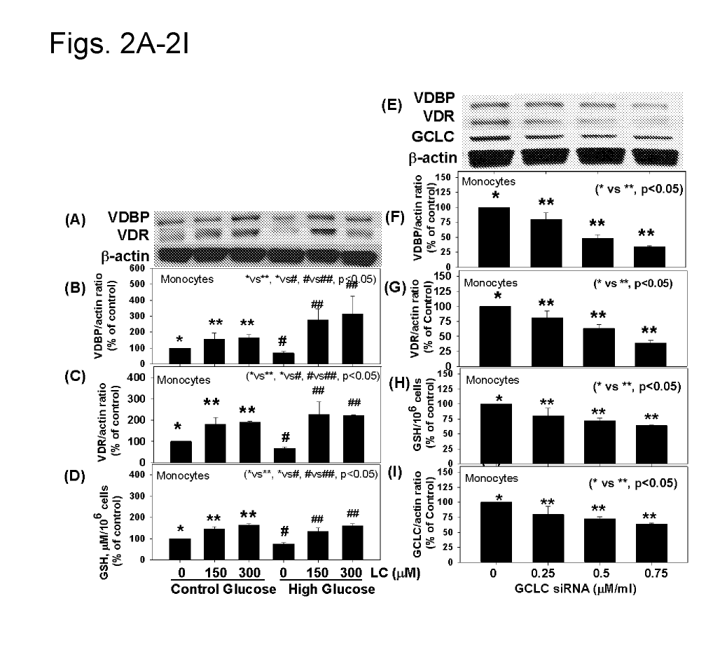

As shown in FIGS. 2A-2I, treatment with high glucose caused a significant decrease in vitamin D binding protein and vitamin D receptor protein expression and glutathione levels in THP-1 human monocytes. More specifically, shown in FIGS. 2A-2D on the left of the page are the effect of L-cysteine supplementation on vitamin D binding protein and vitamin D receptor protein expression (FIG. 2A), vitamin D binding protein/actin ratio (FIG. 2B), vitamin D receptor/actin ratio (FIG. 2C), and glutathione levels (FIG. 2D) in control and high-glucose treated THP-1 monocytes. Shown in FIGS. 2E-2I on the right side of the page is the effect of glutathione deficiency on vitamin D binding protein, vitamin D receptor, and glutamate cysteine ligase catalytic unit-protein expression (FIG. 2E) and vitamin D binding protein/actin ratio (FIG. 2F), vitamin D receptor/actin ratio (FIG. 2G), glutathione (FIG. 2H) and glutamate cysteine ligase catalytic unit knock-down level (FIG. 2I) in THP-1 monocytes treated with different concentrations of glutamate cysteine ligase catalytic unit-siRNA. Values are Mean ±SE (n=4). This data evidences that supplementation with L-cysteine, a precursor of glutathione, results in a significant up-regulation of vitamin D binding protein and vitamin D receptor in monocytes exposed to both control and high glucose concentrations (FIGS. 2B and 2C). Glutathione is formed from L-cysteine by the enzymatic action of glutamate cysteine ligase catalytic unit. To determine whether glutathione has a direct effect on vitamin D binding protein and vitamin D receptor expression, glutathione deficiency was induced by knocking down glutamate cysteine ligase catalytic unit using antisense-mRNA. Increasing the dose of glutamate cysteine ligase catalytic unit-antisense caused increased levels of glutamate cysteine ligase catalytic unit-knock down (FIG. 2I) and glutathione deficiency (FIG. 2H), as well as a decrease in the expression of vitamin D binding protein (FIG. 2F) and vitamin D receptor (FIG. 2G), showing that glutathione deficiency impairs the vitamin D binding protein and vitamin D receptor protein expression in monocytes. There was no change in cell viability as a result of any of the treatments. This data evidences that L-cysteine supplementation upregulates the vitamin D binding protein and vitamin D receptor protein expression mediated by the glutathione in monocytes.

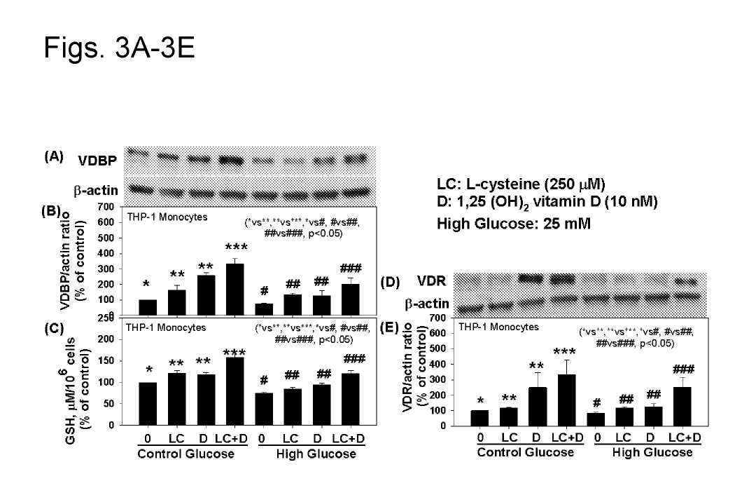

As shown in FIGS. 3A-3E, the administration of vitamin D along with

L-cysteine resulted in a significantly greater up-regulation of both vitamin D binding protein (FIG. 3B) and vitamin D receptor (FIG. 3E) expression compared to treatment with vitamin D alone in THP-1 monocytes. FIGS. 3A-3C on the left side of the page show the effect of L-cysteine and vitamin D supplementation on vitamin D binding protein expression (FIG. 3A), vitamin D binding protein/actin ratio (FIG. 3B), and glutathione levels (FIG. 3C) in control and high-glucose treated THP-1 monocytes. FIGS. 3D-3E on the right side of the page show the effect of L-cysteine and vitamin D supplementation on the vitamin D receptor protein expression (FIG. 3D), and vitamin D receptor/actin ratio (FIG. 3E) in control and high-glucose treated THP-1 monocytes. Values are Mean±SE (n=4). Glutathione levels were also significantly higher in cells treated with vitamin D plus L-cysteine compared to cells treated with only vitamin D or L-cysteine alone (FIG. 3C). The increase in vitamin D binding protein and vitamin D receptor expression caused by vitamin D was lower under high-glucose conditions, which evidences that uncontrolled glycemia in diabetes causes impairment in the metabolic actions of vitamin D. This data presents a path to treat and potentially correct the impaired status of vitamin D and its action in patients with type 2 diabetes via supplementation of vitamin D in combination with L-cysteine, such combination alone or in addition to other diabetes medications preferably in a single pill, shot, or other therapeutic delivery, or spaced into separate therapeutic deliveries.

With regard to glutathione, vitamin D binding protein, and vitamin D status in diabetes, the blood levels of vitamin D binding protein, vitamin D, and glutathione are lower in individuals with diabetes. Elevated urinary loss of vitamin D binding protein and decreased reabsorption in proximal tubule by megalin/Dab2, have been observed by the inventors as one possible factor contributing to the lower vitamin D binding protein and vitamin D levels in individuals with diabetes. The vitamin D binding protein downregulation by high glucose observed by the inventors evidences that uncontrolled glycemia also contributes to lower vitamin D binding protein and vitamin D levels in diabetes. The inventers observed that the decrease in vitamin D binding protein and glutathione levels associated with diabetes is greater in African American diabetic patients than that observed in Caucasian diabetic patients. The inventors disclose for the first time that a significant positive correlation exists between the blood levels of L-cysteine and glutathione and those of vitamin D binding protein and vitamin D in African American type 2 diabetes patients. The inventors' cell culture studies demonstrate first, that high glucose exposure can cause a significant decrease in the vitamin D binding protein and vitamin D receptor protein expression and glutathione levels in monocytes; second, vitamin D binding protein and vitamin D receptor were significantly upregulated by L-cysteine, a precursor of glutathione; and third, that the deficiency of glutathione accomplished by glutamate cysteine ligase catalytic unit knockdown resulted in a significant decrease in vitamin D binding protein and vitamin D receptor expression. This evidences that glutathione status influences expression of vitamin D binding protein and vitamin D receptor in monocytes. As described, treatment with vitamin D and L-cysteine together resulted in significantly greater upregulation of glutathione and vitamin D binding protein and vitamin D receptor in monocytes exposed to high glucose in comparison to treatment with vitamin D alone. This finding provides evidence for a link between vitamin D binding protein and vitamin D with that of glutathione status. Cellular actions influenced by circulating levels of vitamin D are regulated by the vitamin D receptor.

The inventors' experiments showed that L-cysteine plus vitamin D raises blood levels of vitamin D binding protein and vitamin D, especially in African Americans. The inventors are aware that hyperglycemia and diabetes are associated with increased oxidative stress. African Americans are at a higher risk for impaired glutathione status due to their nearly 11% incidence of G6PD deficiency and 6-7% incidence of sickle cell trait, a disease associated with elevated oxidative stress and reduced glutathione. The inventors disclosed herein a link between glutathione status and those of vitamin D binding protein and vitamin D blood levels. Glutathione is a physiological antioxidant, a co-factor of many enzymes, and plays an important role in cellular processes. L-cysteine can also have a direct effect on post-translational modification or S-glutathionylation of proteins, which can cause modification of structure and function and thereby prevention of sulfhydryl overoxidation and proteolysis. L-cysteine can also boost the protection provided against the harm caused by oxidative signaling events. Vitamin D receptor is a potential antioxidant. Vitamin D receptor expression is regulated by physiological factors and hormones including Calcium/Ca²⁺ and 1,25 (OH)₂-D₃. The experimental results showing that L-cysteine upregulates vitamin D binding protein and vitamin D receptor evidences that L-cysteine supplementation stimulates both circulating levels of vitamin D binding protein and vitamin D and the overall efficacy of vitamin D. The discovery of this novel link among the statuses of glutathione, vitamin D binding protein, and vitamin D enable the practice of new therapeutic approaches based on L-cysteine supplementation coupled with lower vitamin D doses to be used as an adjuvant therapy in reducing the vitamin D deficiency and associated health hazards in the African-American population in particular, and in human and mammalian populations in general.

Adult patients with type 2 diabetes were included in the experiments.

All patients who gave written informed consent according to the protocol approved by the Institutional Review Board were invited to return to have blood drawn after fasting overnight. Non-diabetic healthy subjects were also enrolled from siblings of patients or from workers at LSUHSC. Patients were excluded if they had any history of cardiovascular disease, sickle cell disease, treatment with insulin, or metabolic disorders, including uncontrolled hypertension, hypothyroidism, or hyperthyroidism. Patients were excluded if they showed signs of significant hepatic dysfunction, defined as any underlying chronic liver disease or liver function tests greater than 1.5 times the upper limit of normal, or renal dysfunction, defined as a serum creatinine value greater than 1.5 mg /dL. Women with a positive pregnancy test or those nursing infants were also excluded. Subjects were excluded who were taking any supplemental vitamins or herbal products. Blood was drawn after an overnight fast (8 hours). Serum tubes containing blood for chemistry profiles and EDTA-blood tubes for HbA_{1C} and CBC were promptly delivered to the LSUHSC clinical laboratories. Additional EDTA-blood was brought to the research laboratory and the resulting plasma was stored at −80° C. for analyses of the biochemical parameters.

With regard to the L-cysteine, vitamin D, and high glucose treatment and glutamate cysteine ligase catalytic unit knock down in THP-1 monocytes, the THP-1 human monocytic cell line purchased from ATCC (Manassas, Va.) was cultured and maintained in complete RPMI 1640 medium. Pretreatment of the cells, maintained at 1×10⁶/mL of media, was done for 2 hours with L-cysteine (0-300 μM), then 24 hours with 1, 25-(OH)₂-D₃, followed by treatment for 24 hours with high glucose (25 mM). Control cells were treated with mannitol, which is considered an osmolarity control. To investigate the role of glutathione, siRNA specific for glutamate cysteine ligase catalytic unit (the catalytic component of the enzyme) was used to knock down the enzyme and induce glutathione deficiency. Complexes of glutamate cysteine ligase catalytic unit siRNA (Santa Cruz Biotechnology) and lipofectamine (Invitrogen) were allowed to form in culture flasks in serum free media, to which cells suspended in serum free media were added. After 24 hours complete media with serum was added and the cells were then treated as described in the figures.

With regard to the vitamin D, vitamin D binding protein, glutathione, and L-cysteine cell viability assays and immunoblotting, plasma levels of 25-OH-vitamin D were determined using an ELISA kit (Eagle Biosciences, Nashua, NH) and those of vitamin D binding protein using the kit from Alpco (Salem, NH). 25-hydroxy vitamin D (calcidiol) blood test is used to determine how much vitamin D is in the body. The blood concentration of calcidiol is considered an standard indicator of vitamin D status. The kit includes polyclonal antibodies that detect total vitamin D binding protein levels. Levels of glutathione and L-cysteine were determined using HPLC. Details of immunoblotting and cell viability are similar to those given in the publication Vitamin D and 1-cysteine levels correlate positively with glutathione and negatively with insulin resistance levels in the blood of type 2 diabetic patients. Eur J Clin Nutr 68: 1148-1153, 2014 by Jain S K, Micinski D, Huning L, Kahlon G, Bass P F, and Levine SN—such described methods incorporated by reference herein. The antibodies for glutamate cysteine ligase catalytic unit (73 KD), vitamin D binding protein (52 KD), and vitamin D receptor (48 KD) were purchased from Abcam (Cambridge, Mass.).

All chemicals were purchased from Sigma Chemical Co. (St. Louis, Mo.) unless otherwise mentioned. Data were analyzed using ANOVA with Sigma Stat. Body weight was used as additional independent variable to determine regression analyses and p value using Sigma Stat software (SPSS, Chicago, Ill.). A p value of less than 0.05 for a statistical test was considered significant.

### EXAMPLE 2

Cell Culture, L-Cysteine, and High Glucose (HG) Treatment of Hepatocytes

The FL83B hepatocyte cell line was purchased from American Type Culture Collection (ATCC, Manassas, Va., USA). FL83B hepatocytes were cultured and maintained in F-12K complete medium. Pretreatment of the cells, maintained at a concentration of 1×10⁶/mL of media, was done for 2 h with L-cysteine (0-300 μM), followed by treatment for 22 h with HG (25 mM). Control cells were treated with mannitol, which is considered an osmolarity control in some experiments. After 24 h, complete media with serum was added and the cells were then treated as described in the figures.

Animal Studies

The animal protocol was approved by the institutional Animal Welfare Committee (P-15-006). All procedures performed were in accordance with the ethical standards of the institution. Male Zucker diabetic fatty rats at an age of 5 weeks and weighing about 200-220 g were purchased from Charles River (Wilmington, Mass., USA). The animals were allowed to acclimate to the environmental and handling conditions for 2 days. Computer generated randomization was used to divide rats into two groups, after which they were housed and labelled in individual cages. After overnight fasting they were weighed and tested for hyperglycemia by measuring their blood glucose concentration. Blood was collected via tail incision and the blood glucose levels were measured using an Advantage Accu-chek glucometer (Boehringer Mannheim Corp., Indianapolis, Ind., USA). One group of rats was labeled as diabetic controls and gavaged with saline alone. Rats in the other group were labelled as the L-cysteine (LC) group and supplemented with 1 mg L-cysteine/kg body weight daily by oral gavage. A third group included in the study, called the control group, consisted of male Sprague Dawley (SD) rats who were also gavaged daily with saline. Rats in all groups were given an equal volume of saline vehicle or L-cysteine daily for 8 weeks by oral gavage using 20G feeding needles (Popper and Sons, New Hyde Park, N.Y., USA). Blood glucose and body weight were monitored weekly in all rats. Based on any change in their weights the L-cysteine supplementation dose was adjusted accordingly every week to maintain a similar dose per Kg BW over the entire period of the study.

All Zucker diabetic fatty rats were fed a high calorie Purina 5008 lab chow diet (Charles River, Wilmington, Mass., USA). This diet contained 3.4 IU vitamin D₃ per gram diet. Sprague Dawley rats were fed a standard 8640 lab chow diet, which contained 3 IU vitamin D₃ per gram diet (Harlan, Indianapolis, Ind., USA); both Zucker diabetic fatty and Sprague Dawley rats were maintained at 22 ±2° C. with 12:12-h light/dark cycles. Food intake of all rats was monitored. The control group contained six rats. There were also six rats each in the placebo and L-cysteine-supplemented diabetic rat groups. At the end of 8 weeks the rats were fasted overnight and euthanized by exposing them to halothane (2-bromo-2-chloro-1,1,1-trifluoroethane). Blood was drawn with a 191/2 gauge needle via cardiac puncture into vacutainer tubes. Aliquots of blood collected from all rats were sent to the clinical laboratory of LSUHSC-Shreveport for blood chemistry profiles, including liver and renal function and red blood cell counts. Plasma was isolated in a 4° C. centrifuge at 3000 rpm for 10 min from blood collected into EDTA tubes. Rat livers were perfused using cold saline. Once extracted, they were labeled appropriately, and stored at −80° C.

Quantitative PCR in Rat Liver Lysates

About 50 mg of liver tissue was weighed and homogenized in 1 mL TRIzol reagent (Invitrogen, Grand Island, N.Y., USA). The RNA extraction was performed according to the instructions provided. The concentration and quality of the extracted RNA were determined on a NanoDrop spectrophotometer (Thermo Scientific, Pittsburgh, Pa., USA). A High Capacity RNA-To-cDNA kit (Invitrogen) was used to synthesize cDNA. QPCR was performed using a 7900HT Real Time PCR system and software (Applied Biosystems, Grand Island, NY, USA) using the FAM-labeled primer/probe set Rn00561256_m1 for vitamin D binding protein (also called GC), Rn00690616_m1 for vitamin D receptor, Rn01754615 ml for CYP2R1, and Rn01775763_g1 for GAPDH (Invitrogen), respectively. The relative fold change of mRNA was calculated using the relative quantification (ΔΔCT) method.

25(OH) Vitamin D, Vitamin D Binding Protein, and Cytokine Assays

Plasma levels of 25(OH) vitamin D, commonly used to de were determined using an ELISA kit (Eagle Biosciences, Nashua, N.H., USA). Plasma vitamin D binding protein quantification was carried out using a kit purchased from Alpco (Salem, N.H., USA). The kit includes polyclonal antibodies that detect total vitamin D binding protein levels. The cytokines MCP-1 (R&D Systems Inc., Minneapolis, Minn., USA) and CRP (Alpco) were determined using the sandwich ELISA method and commercially available kits. In the cytokine assay, control samples were analyzed each time to check the variation from plate to plate on different days of analysis. Protocols as given in the manufacturer's kit were followed using appropriate controls and standards.

Glutathione, L-Cysteine, Cell Viability Assays, and Immunoblotting

Levels of glutathione and L-cysteine were determined using HPLC. In cell culture studies, the whole cell suspension was processed for the glutathione assay. Cell viability was determined using the Alamar Blue method (Alamar Biosciences, Sacramento, Calif., USA). Details of immunoblotting are similar to those known in the art. The antibodies for vitamin D binding protein (52 KD), β-Actin (40 KD), glutamate-cysteine ligase catalytic subunit (GCLC) (75 KD), and vitamin D receptor (48 KD) were purchased from Abcam (Cambridge, Mass., USA). The intensity of each immunoblotting band was measured using the histogram tool of Adobe Photoshop CS5.

Reactive Oxygen Species Assay

Hepatocytes were lysed after treatment and reactive oxygen species were measured in the cell lysates using 20 μM of the oxidant-sensitive probe dicholorodihydrofluorescein diacetate (H₂DCFDA, Sigma Chemical Co., St. Louis, Mo., USA). Liver lysate aliquots (20 μg) were used to measure reactive oxygen species with H₂DCFDA. Details of the reactive oxygen species assay are as known in the art.

GCLC siRNA Transection

GCLC siRNA was purchased from Santa Cruz Biotechnology, Inc. (Dallas, Tex., USA), Catalogue number, sc-41979. The y-GCLC siRNA used is a pool of three different siRNA duplexes. Sequences of siRNA oligonucleotides used is given in Supporting Information Material. Different concentrations of siRNA (0-200 nM) were used to knockdown GCLC using transfection reagent (lipofectamine, Invitrogen) as known in the art.

Quantitative PCR in Hepatocytes

mRNA extraction and cDNA synthesis were done as described in the previous section. The following primers were used for hepatocytes: vitamin D binding protein (also called GC) (Mm04243540_m1), vitamin D receptor (Mm00437297_m1), GCLC (Mm00802655_m1), and GAPDH (Mm03302249_g1) and were purchased from Invitrogen. Sequence of oligonucleotides of primers used is given in Supporting Information Material. The relative fold change of mRNA was calculated using the relative quantification (ΔΔCT) method and then normalized to % control for representation.

All chemicals were purchased from Sigma Chemical Co. (St. Louis, Mo., USA) unless otherwise mentioned. Data from cell culture and rat studies were analyzed using ANOVA with Sigma Stat software (SPSS, Chicago, Ill., USA). A p value of less than 0.05 for a statistical test was considered significant.

Effect of L-Cysteine Supplementation on Vitamin D Binding Protein and Vitamin D Receptor Expression in Hepatocytes

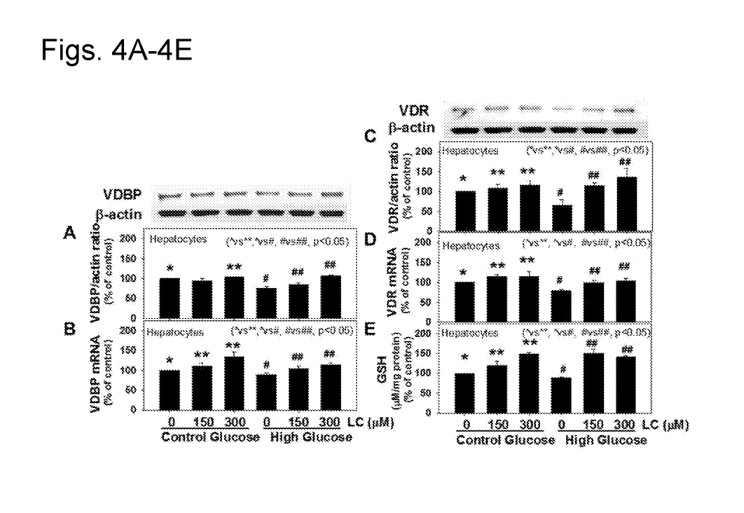

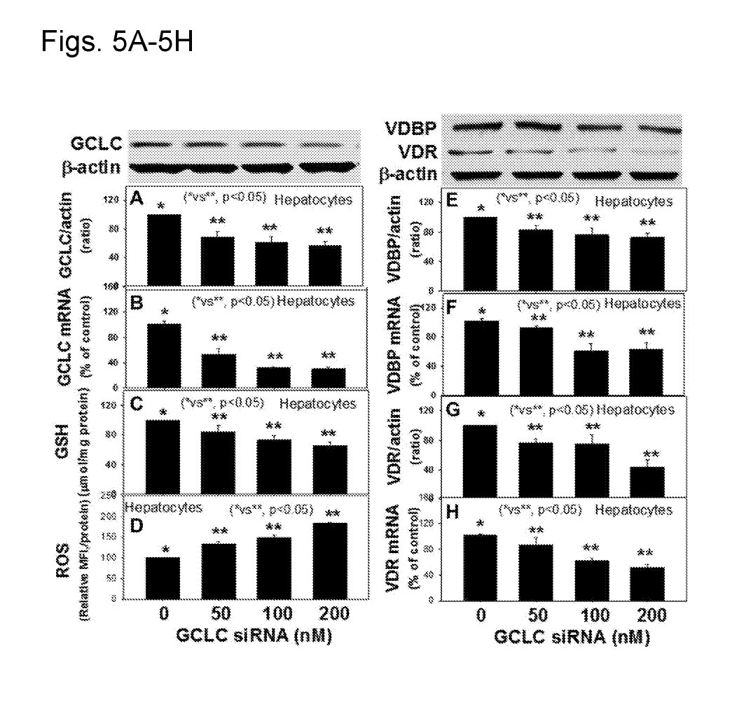

FIGS. 4A-4E illustrate the effect of L-cysteine supplementation on glutathione and vitamin D binding protein and vitamin D receptor in normal and high glucose (HG)-treated hepatocytes. High glucose treatment caused a significant decrease in both the protein expression and mRNA levels of vitamin D binding protein and vitamin D receptor in hepatocytes; L-cysteine supplementation resulted in upregulation of both protein expression and mRNA levels of vitamin D binding protein (FIGS. 4A and 4B) and vitamin D receptor (FIGS. 4C and 4D) in hepatocytes cultured with high glucose. There was a significant decrease in glutathione levels (FIG. 4E) after high glucose treatment, which was prevented by supplementation with L-cysteine, a precursor of glutathione, in hepatocytes. glutathione deficiency was induced by knocking down GCLC using antisense-mRNA to determine whether glutathione has a direct effect on vitamin D binding protein and vitamin D receptor expression in hepatocytes. FIGS. 5A-5H illustrate the effect of GCLC-antisense on GCLC knockdown (GCLC-KD) (FIGS. 5A and 5B), decrease in glutathione (FIG. 5C), and an increase in reactive oxygen species (FIG. 5D) levels, as well as a decrease in the vitamin D binding protein (FIGS. 5E and 5F) and vitamin D receptor (FIGS. 5G and 5H) levels. This suggests that glutathione deficiency can impair the vitamin D binding protein and vitamin D receptor protein and mRNA expression in hepatocytes.

Effect of L-Cysteine Supplementation on Vitamin D Binding Protein, 25(OH) Vitamin D, Glutathione, MCP-1, and CRP Levels in Zucker Diabetic Fatty Rats

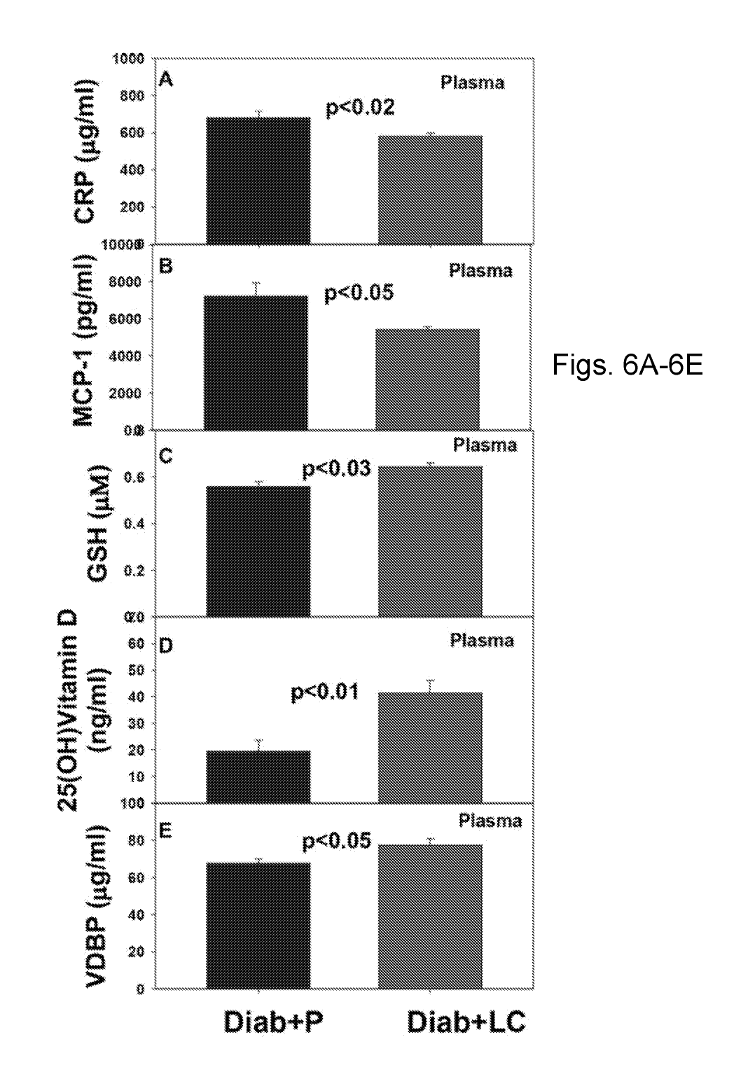

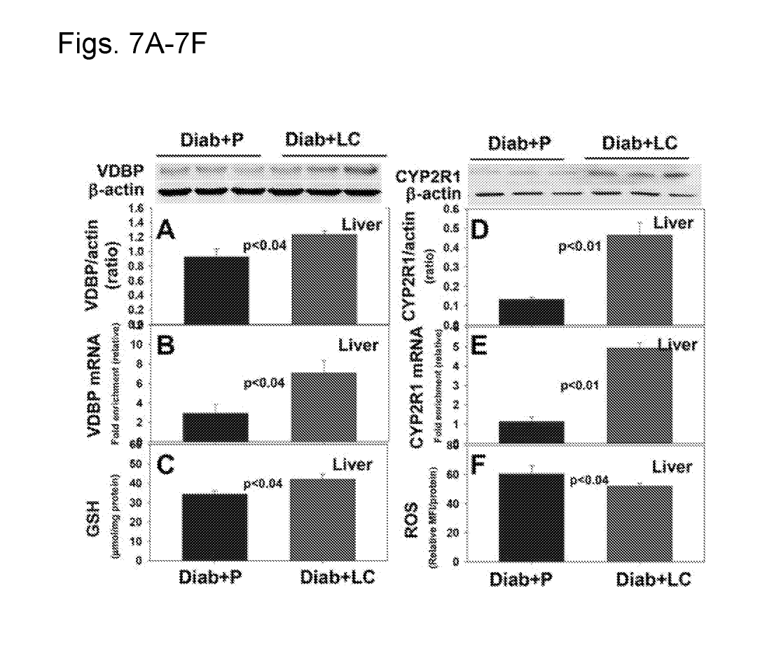

Cell culture studies suggest that L-cysteine upregulates the vitamin D binding protein and vitamin D receptor protein expression mediated by the glutathione status in monocytes. This led to our hypothesis that supplementation with L-cysteine, a precursor of glutathione, may boost the blood levels of vitamin D binding protein and 25(OH) vitamin D, and its metabolic actions in vivo in diabetic rats. FIGS. 6A-6E show the effect of L-cysteine or placebo supplementation on blood levels of vitamin D binding protein, 25(OH) vitamin D, glutathione, and biomarkers of inflammation in Zucker diabetic fatty rats. Blood levels of both vitamin D binding protein (FIG. 6E) and 25(OH) vitamin D (FIG. 6D) were significantly higher in L-cysteine-supplemented compared with placebo-supplemented diabetic rats. L-cysteine-supplemented diabetic rats also showed an increase in plasma glutathione (FIG. 6C) and a decrease in MCP-1 (FIG. 6B) and CRP (FIG. 6A). FIGS. 7A-7F show a significantly higher protein and mRNA expression of vitamin D binding protein (FIGS. 7A and 7B) and vitamin D 25-hydroxylase (CYP2R1) (FIGS. 7D and 7E) levels in the livers of rats supplemented with L-cysteine compared to those supplemented with placebo; glutathione levels (FIG. 7C) were significantly higher and reactive oxygen species (FIG. 7F) levels were significantly lower in the livers of L-cysteine-supplemented rats. The livers of L-cysteine-supplemented rats showed higher levels of expression of protein (p=0.17) and mRNA (p=0.30) for vitamin D receptor, but these values were not statistically significant in comparison to those of placebo-supplemented rats. This suggests that L-cysteine supplementation can increase blood levels of vitamin D binding protein and 25(OH) vitamin D, and also decrease inflammation biomarkers in vivo in type II diabetes rats.

Table 2 shows no change in the blood levels of biomarkers of liver and kidney function tests, including blood cell counts or serum calcium, between L-cysteine and placebo groups, suggesting that L-cysteine supplementation is safe. There was no difference in daily food intake between L-cysteine-supplemented and placebo-supplemented diabetic rats. There were significantly lower body weights and an increase in plasma L-cysteine levels among L-cysteine-supplemented rats compared with those in the placebo group. Total cholesterol and triglyceride levels also did not differ between L-cysteine-supplemented and placebo-supplemented rats. This suggests that no negative side effects result from L-cysteine supplementation.

TABLE 2
The effect of L-cysteine supplementation on body weight,
plasma lipids, and liver and kidney function tests in
Zucker diabetic fatty rats. Rats were supplemented with
placebo (saline) or L-cysteine (1 mg/kg BW) daily gavage
for 8 weeks. Values are mean ± SE. Values marked *
are significantly different (p < 0.05)

| | Diabetic + P | Diabetic + LC |
|---|---|---|
| N | 6 | 6 |
| Body weight (g) | 371 ± 3.9 | 346 ± 6.3* |
| Food intake (g/day) | 38 ± 1.2 | 37.1 ± 1.0 |
| AST (IU/L) | 173 ± 32 | 181 ± 19 |
| ALT (IU/L) | 99.6 ± 6.4 | 95.3 ± 13.1 |
| ALP (IU/L) | 24.0 ± 4.9 | 22.5 ± 3.1 |
| Creatinine (mg/dL) | 0.4 ± 0.0 | 0.4 ± 0.0 |
| RBC (10⁶/μL) | 9.5 ± 0.15 | 9.04 ± 0.07 |
| Hemoglobin (g/dL) | 16.0 ± 0.23 | 15.42 ± 0.14 |
| L-cysteine (μM) | 173.4 ± 14.2 | 218.8 ± 7.7* |
| Calcium (mg/dL) | 10.1 ± 0.3 | 9.9 ± 0.4 |
| Total cholesterol (mg/dL) | 188.9 ± 7.0 | 171 ± 11.5 |
| Triglycerides (mg/dL) | 571 ± 29 | 509 ± 55 |

Vitamin D Binding Protein, Vitamin D Receptor, and CYP2R1 Status in Liver of Diabetic Rats

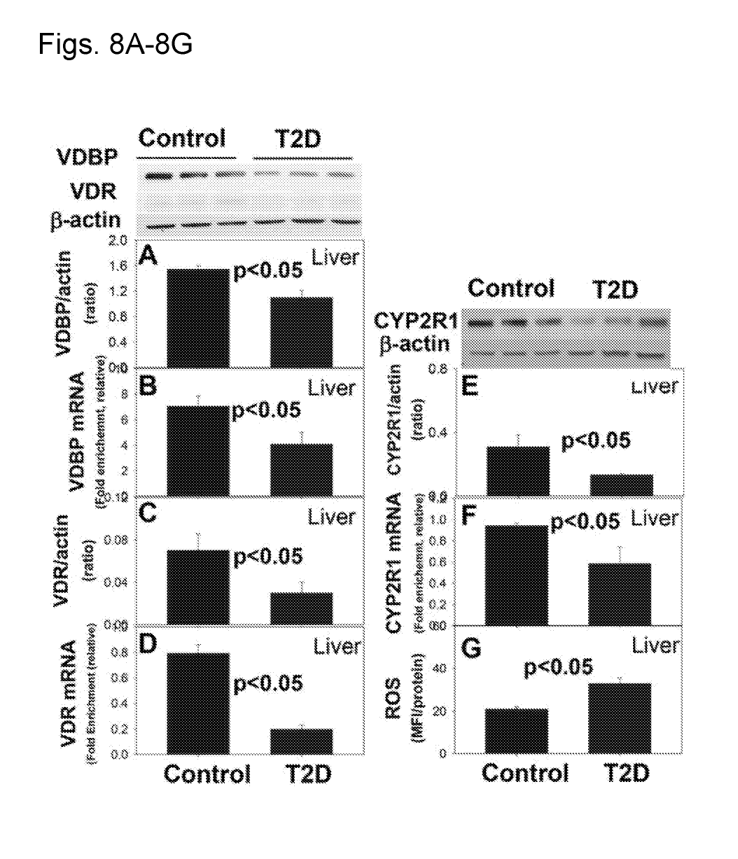

We investigated vitamin D binding protein and vitamin D receptor status in the liver to investigate whether hyperglycemia in vivo also has any effect on the vitamin D binding protein and vitamin D receptor downregulation similar to that observed in high glucose-treated cultured hepatocytes. FIGS. 8A-8D and 8G illustrate that protein and mRNA expression of vitamin D binding protein (FIGS. 8A and 8B) and vitamin D receptor (FIGS. 8C and 8D) is significantly lower, and reactive oxygen species levels are significantly higher (FIG. 8G), in the livers of Zucker diabetic fatty rats compared with those of age-matched normal rats. FIGS. 8E and 8F also show lower levels of both protein and mRNA expression of CYP2R1 in type 2 diabetic rats compared with those in normal rats. Rats in each group were 14 weeks old; blood glucose levels were significantly higher (349±19 mg %) in diabetic rats compared with those in normal rats (162±8 mg %). This suggests that level of vitamin D binding protein and vitamin D receptor expression were significantly reduced both in hepatocytes treated with high glucose (FIG. 4) and in the liver of diabetic rats. This suggests that uncontrolled glycemia in diabetes may play a role in the lower blood levels of vitamin D binding protein seen in diabetic rats.

### Discussion

Epidemiological studies suggest a positive association between better health outcomes and higher blood levels of 25(OH) vitamin D. This study reports that L-cysteine supplementation can boost circulating levels of 25(OH) vitamin D and its efficacy, mediated by an increase in glutathione and vitamin D binding protein levels in Zucker diabetic fatty rats, a model of type 2 diabetes.

Vitamin D binding protein is primarily synthesized and secreted by the liver. vitamin D binding protein, also called GC, is a 52-59 kDa monomeric glycoprotein with a short half-life of 2.5-3 days compared with the 1-2 month half-life of 25(OH) vitamin D. Studies of humans with a genetic mutation for vitamin D binding protein and vitamin D binding protein-knockdown mice demonstrate low plasma levels of 25(OH) vitamin D. Genetic variations in vitamin D binding protein are known to influence 25(OH) vitamin D blood levels in response to vitamin D supplementation. vitamin D binding protein plays a major role in transporting vitamin D and 25(OH) vitamin D to various tissues and participating in their conversion to 1,25(OH)₂ vitamin D₃, an active form of vitamin D. Blood concentrations of vitamin D binding protein has been evidenced to be positively related to the half-life of circulating 25(OH) vitamin D. Increasing vitamin D binding protein availability can potentially increase 1,25(OH)₂ vitamin D₃ levels and the metabolic actions of 25(OH) vitamin D. All cells in the body, including hepatocytes, have receptors known as vitamin D receptor. The biological response to 1,25(OH)₂ vitamin D₃ is directly related to the vitamin D receptor content of target tissues and vitamin D receptor expression is regulated by physiological factors including calcium, 1,25(OH)₂ vitamin D₃, and vitamin D binding protein. This evidences that the higher circulating levels of 25(OH) vitamin D could be due to the upregulation of vitamin D binding protein and vitamin D 25 hydroxylase, as seen in the liver and blood of L-cysteine-supplemented Zucker diabetic fatty rats compared with those of placebo-supplemented rats consuming a similar diet, could be due to the upregulation of vitamin D binding protein status. The decrease in inflammatory biomarkers could be due to improved metabolic actions or efficacy of 25(OH) vitamin D mediated by the upregulation of vitamin D receptor in L-cysteine-supplemented rats.

The inventors are aware that diabetes is associated with lower blood levels of vitamin D binding protein, 25(OH) vitamin D, L-cysteine, and glutathione. Glutathione is a physiological antioxidant, a co-factor of many enzymes, and plays an important role in a multitude of cellular processes. L-cysteine can also have a direct effect on post-translational modification or S-glutathionylation of proteins, which can cause modification of structure and function and thereby provide protection against oxidative signaling events. Elevated oxidative stress in various tissues from uncontrolled hyperglycemia may cause increased L-cysteine utilization and the lower levels seen in diabetes. Glutathione is formed from L-cysteine by the enzymatic action of glutamate-cysteine ligase (GCLC). L-cysteine supplementation and an improvement in glutathione status is potentially useful for prevention of oxidative stress and insulin resistance. Renal dysfunction and increased urinary excretion of vitamin D binding protein and 25(OH) vitamin D are implicated in the 25 (OH) vitamin D deficiencies associated with diabetes. Glutathione deficiency, induced by GCLC knockdown, resulted in the downregulation of vitamin D binding protein and vitamin D receptor in hepatocytes, which evidences that an improvement in the vitamin D binding protein and 25 (OH) vitamin D statuses would be associated with an improvement in the glutathione levels in L-cysteine-supplemented rats. The beneficial effect of L-cysteine on biomarkers of vascular inflammation in diabetic rats may result from a beneficial effect on lowered oxidative stress. The increases in blood levels of glutathione, vitamin D binding protein, and 25(OH) vitamin D in L-cysteine-supplemented diabetic rats, along with data from cell culture studies, evidence a link between glutathione status and those of vitamin D binding protein and 25(OH) vitamin D blood levels in diabetes.

There are contrary views as to the importance of the free form of 25(OH) vitamin D. Some suggest that 25(OH) vitamin D that is not bound to the vitamin D binding protein or the free form of 25(OH) vitamin D is important for the biological actions of 25(OH) vitamin D. However, others challenge this view. 25(OH) vitamin D bound to the vitamin D binding protein is, in fact, the form that participates in 1,25(OH)₂ vitamin D₃ synthesis and in the regulation of parathyroid hormone. It is possible that both 25(OH) vitamin D bound to vitamin D binding protein and free 25(OH) vitamin D play important roles in the metabolic actions and efficacy of circulating 25(OH) vitamin D under different physiological and pathological conditions.

This study has evidenced a link between the status of 25(OH) vitamin D and vitamin D binding protein, and that of glutathione and L-cysteine, using a cell culture model and in diabetic rats.

Deficiency in glutathione and glutathione generating enzymes is linked to obesity in rodents and humans. Decreased glutathione accelerates adipogensis in adipocytes. The inventors' experiments also showed an increase in glutathione levels and a decrease in body weight gain in L-cysteine-supplemented diabetic rats compared with placebo. Reduction in oxidative stress can mediate the upregulation of both the insulin dependent and insulin independent signaling cascades, GLUT4, and glucose metabolism, and lower insulin resistance, thereby contributing to reduction in weight gain in L-cysteine-supplemented diabetic rats. This indicates that an improvement in glutathione status is a potentially useful therapeutic target for prevention of oxidative stress and weight gain. The inventors also show that L-cysteine supplementation appears to regulate glutathione and energy expenditure and weight gain, and that levels protein and mRNA expression of vitamin D binding protein, vitamin D receptor, and vitamin D 25-hydroxylase were significantly reduced in liver of diabetic rats. This evidences that uncontrolled glycemia may also contribute to the lower blood levels of vitamin D binding protein observed in addition to the excess urinary loss of vitamin D binding protein in diabetic rats. The changes seen in vitamin D binding protein and vitamin D receptor in livers of diabetic rats are likely to be from the hyperglycemia, because a similar effect, lower expression of vitamin D binding protein and vitamin D receptor, was seen in high-glucose treated hepatocytes compared with control cells.

This study evidences a link between lower blood levels of vitamin D binding protein and 25 (OH) vitamin D with those of lower glutathione, and that increases in glutathione levels as the result of L-cysteine supplementation can upregulate vitamin D binding protein and 25(OH) vitamin D status. It appears that supplementation with L-cysteine increases blood levels of glutathione and prevents the excess oxidative stress/reactive oxygen species (ROS) associated with type II diabetes. The upregulation of glutathione and decrease in reactive oxygen species results in upregulation of vitamin D binding protein, vitamin D 25-hydroxylase and thereby increased blood levels of 25(OH) vitamin D, and its metabolic actions. The potential mechanism for the increased blood level of 25(OH) vitamin D could be normalization of the status of vitamin D binding protein, resulting in the better transport of cholcalciferol by the vitamin D binding protein for its hydroxylation. Another potential mechanism could involve the up-regulation of protein expression and activity of 25-vitamin D hydroxylase by either increased levels of glutathione or lower oxidative stress in L-cysteine-supplemented diabetic rats.

25(OH) vitamin D deficiency and an excessive rate of diabetes have both become epidemic worldwide. Vitamin D supplementation is widely recommended, but higher doses of vitamin D supplementation are needed to achieve normal circulating 25(OH) vitamin D levels, and some patients do not respond well to supplementation. This study reports that L-cysteine supplementation can boost circulating levels of 25(OH) vitamin D and its efficacy, mediated by an increase in glutathione, vitamin D binding protein, and vitamin D 25-hydroxylase levels in Zucker diabetic fatty rats. This invention could have a tremendous impact on the practice of medicine and is a novel approach that combined vitamin D and L-cysteine supplementation be used, instead of supplementation with vitamin D alone, to optimize 25(OH) vitamin D levels, thus helping to address and possibly prevent the health hazards associated with diabetes.

### Supplemental Material

Oligonucleotide Sequence for GCLC siRNA

GCLC siRNA was purchased from Santa Cruz Biotechnology, Inc. (Dallas, Tex.), Catalogue number, sc-41979. γ-GCLC siRNA used is a pool of different siRNA duplexes. (1) sc-41979A: Sense: CGGUAUGACUCAAUAGAUAtt Antisense: UAUCUAUUGAGUCAUACCGtt (2) sc-41979B: Sense: CUAGGGUGAUCCUCUCAUAtt Antisense: UAUGAGAGGAUCACCCUAGtt (3) sc-41979C: Sense: GAAGUACAGUGGAGGUAAAtt Antisense: UUUACCUCCACUGUACUUCtt. All sequences are provided in 5′→3′ orientation.

### Oligonucleotide Sequence for Rat Primers

VDBP/GC (Assay ID—Rn00561256_m1): t cagtttctat tcgaatattc cagcaattac ggacaagctc ctctgccact tttagttggt tacaccaaga g; Exon boundary: 4-5, Assay Location: 520, Amplicon Length: 72.

VDR (Assay ID—Rn00690616_m1): cctga cagatgagga ggtacagcgt aagagggaga tgataatgaa gagaaaagag gaagaggc; Exon boundary: 4-5, Assay Location: 376, Amplicon Length: 63

CYP2R1 (Assay ID—Rn01754615_m): aggacaag ttcataaaga gattgattta attatgggac acgacaggag gccttcttgg gaagacaaat gcaaaatgcc tta; Exon boundary: 3-4, Assay Location: 373, Amplicon Length: 81

GCLC (Rn00689046_m1): tctggagaa ctaatgactg ttgccaggtg gatgagagag tttattgcaa accatcctga ctacaagcaa gaca; Exon boundary: 15-16, Assay Location: 1762, Amplicon Length: 73

GAPDH (Rn01775763_g1): ccaactc agcccccaac actgagcatc tccctcacaa ttccatccca gaccccataa caacaggagg ggcctgggga gccctccctt ctctcgaata ccatcaataa agttcgctgc accctcaaaa aaaaaaaaaa aaaaaaaaaa aaaaaa; Exon boundary: 8-8, Assay Location: 1153, Amplicon Length: 174

### Oligonucleotide Sequence for Mouse Primers

VDBP/GC (Mm04243540_m1): ag gacctcagag ctgtctgtta agtcctgtga aagtgatgctccctttccgg ttcaccctgg aact; Exon: 3-4, Assay Location: 439, Amplicon Length: 66

VDR (Mm00437297_m1): gg agcatgaagc gcaaggccct gttcacctgc cccttcaatg gagattgccg catcaccaag gacaaccggc gacactgcca ggcctgccgg ct; Exon: 3-4, Assay Location: 289, Amplicon Length: 95

GCLC (Mm00802655_m1): ccaag gttgacgaga acatgaaagt ggcccagaag cgagatgctg tcttgcaggg gatgttttat ttcaggaaag acatctgcaa aggcggcaac gctg; Exon: 12-13, Assay Location: 1666, Amplicon Length: 98

GAPDH (Mm99999915_g1): tttg gccgtattgg gcgcctggtc accagggctg ccatttgcag tggcaaagtg gagattgttg ccatcaacga ccccttcatt gacctcaact acatggtcta cat, Exon: 2-3, Assay Location: 117, Amplicon Length: 107

The form of vitamin D that was used in the experiments was vitamin D₃, also known as cholecalciferol. In addition to vitamin D₃, other forms of vitamin D may be used in disclosed treatment, including vitamin D₁, vitamin D₂, vitamin D₃, vitamin D₄, and vitamin D₅ (chemical structures for each or for constituent parts of each shown below), as may hormonally active metabolites thereof, pharmaceutically acceptable salts, solvates, clathrates, stereoisomers, enantiomers or prodrugs thereof, and other pharmaceutically acceptable derivatives thereof.

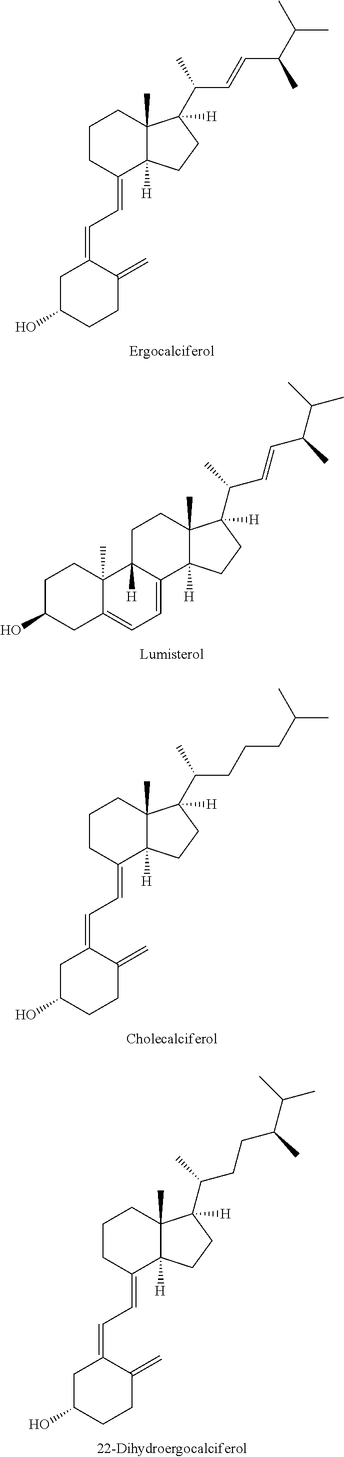

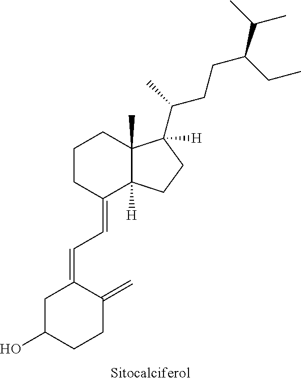 [SMILES: C=C1CCC(O)C/C1=C/C=C1\CCC[C@]2(C)[C@@H]([C@H](C)CC[C@H](CC)C(C)C)CC[C@@H]12]

Forms of L-cysteine that may be used in the disclosed treatment may include pharmaceutically acceptable salts, solvates, clathrates, stereoisomers, enantiomers or prodrugs thereof, and pharmaceutically acceptable derivatives thereof.

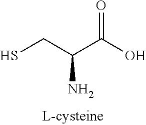

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating one of vitamin D deficiency, diabetes and pre-diabetes in an animal comprising the steps of:
administering a first therapeutic agent and a second therapeutic agent to the animal;
providing the first therapeutic agent is one of vitamin D, a pharmaceutically acceptable salt, solvate, clathrate, stereoisomer, enantiomer or prodrug thereof, and a pharmaceutically acceptable derivative thereof; and
providing the second therapeutic agent is one of L- cysteine, glutathione; glycine, glutamate, and glutathione, or a pharmaceutically acceptable salt, solvate, clathrate, stereoisomer, enantiomer or prodrug thereof, pharmaceutically acceptable derivative thereof, or some combination of thereof.

2. The method of claim 1 further comprising the first therapeutic agent is administered in a dosage of between 100 IU and 1500 IU.

3. The method of claim 1 further comprising the second therapeutic agent is administered in a dosage of between 1 mg and 10 mg per kg of a body weight of the animal.

4. The method of claim 1 further comprising the animal being a human.

5. The method of claim 1 further comprising the step of administering the first and the second therapeutic agent in a single pill or serum.

6. The method of claim 1 further comprising the step of administering a third therapeutic agent.

7. The method of claim 6 wherein the third therapeutic agent is one or more of a sulfonylurea, a biguanide, a meglitinide, a thiazolidinedione, a DPP-4 inhibitor, an SGLT2 inhibitor, an alpha-glucosidase inhibitor, and a bile acid sequestrant, and a pharmaceutically acceptable salt, solvate, clathrate, stereoisomer, enantiomer or prodrug thereof, pharmaceutically acceptable derivative thereof, or some combination of thereof.

8. The method of claim 6 wherein the third therapeutic agent is a sulfonylurea or a pharmaceutically acceptable salt, solvate, clathrate, stereoisomer, enantiomer or prodrug thereof, pharmaceutically acceptable derivative thereof, or some combination of thereof.

9. The method of claim 8 wherein the sulfonylurea is one of glyburide, glimepiride, tolbutamide, tolazamide, chlorpropamide and glipizide or a pharmaceutically acceptable salt, solvate, clathrate, stereoisomer, enantiomer or prodrug thereof, pharmaceutically acceptable derivative thereof, or some combination of thereof.

10. The method of claim 6 wherein the third therapeutic agent is a biguanide from metformin or a pharmaceutically acceptable salt, solvate, clathrate, stereoisomer, enantiomer or prodrug thereof, pharmaceutically acceptable derivative thereof, or some combination of thereof.

11. The method of claim 6 wherein the third therapeutic agent is a meglitinide from one of repaglinide and nateglinide or a pharmaceutically acceptable salt, solvate, clathrate, stereoisomer, enantiomer or prodrug thereof, pharmaceutically acceptable derivative thereof, or some combination of thereof.

12. The method of claim 6 wherein the third therapeutic agent is a thiazolidinedione from one of rosiglitazone and pioglitazone or a pharmaceutically acceptable salt, solvate, clathrate, stereoisomer, enantiomer or prodrug thereof, pharmaceutically acceptable derivative thereof, or some combination of thereof.

13. The method of claim 6 wherein the third therapeutic agent is a DPP-4 inhibitor from one of sitagliptin, saxagliptin, linagliptin, and alogliptin or a pharmaceutically acceptable salt, solvate, clathrate, stereoisomer, enantiomer or prodrug thereof, pharmaceutically acceptable derivative thereof, or some combination of thereof.

14. The method of claim 6 wherein the third therapeutic agent is a SGLT2 inhibitor from one of canagliflozin and dapagliflozin or a pharmaceutically acceptable salt, solvate, clathrate, stereoisomer, enantiomer or prodrug thereof, pharmaceutically acceptable derivative thereof, or some combination of thereof.

15. The method of claim 6 wherein the third therapeutic agent is an alpha-glucosidase inhibitor from one of acarbose and miglitol or a pharmaceutically acceptable salt, solvate, clathrate, stereoisomer, enantiomer or prodrug thereof, pharmaceutically acceptable derivative thereof, or some combination of thereof.

16. The method of claim 6 wherein the third therapeutic agent is a bile acid sequestrant colesevelam or a pharmaceutically acceptable salt, solvate, clathrate, stereoisomer, enantiomer or prodrug thereof, pharmaceutically acceptable derivative thereof, or some combination of thereof.

17. The method of claim 1 further comprising the vitamin D being one or more of vitamin D₁, vitamin D₂, vitamin D₃, vitamin D₄, and vitamin D₅

18. The method of claim 1 wherein the human is African American.

19. A pharmaceutical comprising:
a first and a second therapeutic agent;
the first therapeutic agent being one of vitamin D, a pharmaceutically acceptable salt, solvate, clathrate, stereoisomer, enantiomer or prodrug thereof, pharmaceutically acceptable derivative thereof, or some combination of thereof; and
the second therapeutic agent being one of L- cysteine, glutathione; glycine, glutamate, and glutathione, or a pharmaceutically acceptable salt, solvate, clathrate, stereoisomer, enantiomer or prodrug thereof, pharmaceutically acceptable derivative thereof, or some combination of thereof.

20. The pharmaceutical of claim 19 further comprising a third therapeutic agent, wherein the third therapeutic agent is one or more of a sulfonylurea, a biguanide, a meglitinide, a thiazolidinedione, a DPP-4 inhibitor, an SGLT2 inhibitor, an alpha-glucosidase inhibitor, and a bile acid sequestrant, and a pharmaceutically acceptable salt, solvate, clathrate, stereoisomer, enantiomer or prodrug thereof, pharmaceutically acceptable derivative thereof, or some combination of thereof.
